(19) United States
(12) Patent Application Publication
(10) Pub. No.: US 2009/0192187 A1
(43) Pub. Date: Jul. 30, 2009

(54) DRY POWDER FORMULATION COMPRISING AN ANTICHOLINERGIC DRUG

(75) Inventors: Gaetano BRAMBILLA, Parma (IT); Rossella Musa, Parma (IT); Daniela Cocconi, Parma (IT)

Correspondence Address: OBLON, SPIVAK, MCCLELLAND MAIER & NEUSTADT, P.C., 1940 DUKE STREET, ALEXANDRIA, VA, 22314, US

(73) Assignee: Chiesi Farmaceutici S.p.A., Parma (IT)

(21) Appl. No.: 12/343,769

(22) Filed: Dec. 24, 2008

(30) Foreign Application Priority Data
Jan. 15, 2008 (EP) 08000629.9

(51) Int. Cl. A61K 31/439; A61P 11/00; A61P 11/06

(52) U.S. Cl. 514/305

(57) ABSTRACT

Pharmaceutical formulation in the form of inhalable dry powder comprising particles of a pharmaceutically acceptable salt of 3-[[[(3-fluorophenyl)[(3,4,5-trifluoro phenyl)methyl]amino]carbonyl]oxy]-1-[2-oxo-2-(2-thienyl)ethyl]-1-azoniabicyclo [2.2.2]octane as active ingredient, and particles of a carrier made of a physiologically acceptable pharmacologically-inert material are effective for the prevention and/or treatment of a respiratory disease such as asthma and COPD.

### CROSS REFERENCES TO RELATED APPLICATIONS

This application claims priority to European Patent Application No. 08000629.9, filed on Jan. 15, 2008, which is incorporated herein by reference in its entirety.

### BACKGROUND OF THE INVENTION

1. Field of the Invention

The present invention relates to a dry powder formulations suitable to be administered by inhalation by means of a dry powder inhaler which are useful for the prevention and/or treatment of an inflammatory or obstructive airways disease such as asthma and COPD. The present invention also relates to processes for the preparation of such formulations and inhalers containing such a formulation, and to methods for the prevention and/or treatment of an inflammatory or obstructive airways disease such as asthma and COPD by administering such a formulation.

2. Discussion of the Background

Quaternary ammonium salts acting as muscarinic receptors antagonists are currently used in therapy to induce bronchodilation for the treatment of respiratory diseases, and in particular, inflammatory or obstructive airway diseases such as asthma and chronic obstructive pulmonary disease (COPD).

For treating chronic diseases, it is often desirable to utilize antimuscarinic drugs with a long-lasting effect. This ensures that the concentration of the active substance necessary for achieving the therapeutic effect is present in the lungs for a long period of time, without the need for the active substance to be administered repeatedly and too frequently. In particular, it would be desirable to utilize antimuscarinic drugs which are therapeutically efficacious upon administration by inhalation once a day. In order to fulfill such a requirement, antimuscarinic drugs should exhibit good selectivity for M3 muscarinic receptors, and slow dissociation from them.

Recently it has been reported that tiotropium bromide, the first drug in a new generation of antimuscarinic drugs, exhibits a very slow dissociation from M3 receptors, behaviour thought to account for its long lasting activity. However tiotropium bromide still retains a slow dissociation kinetics for the M2 muscarinic receptors. Since M2 receptors are a major population in the cardiac muscle, a therapy with said drug might be accompanied by undesired cardiac side effects.

The quaternary ammonium salt 3-[[[(3-fluorophenyl)[(3,4,5-trifluoro phenyl)methyl]amino]carbonyl]oxy]-1-[2-oxo-2-(2-thienyl)ethyl]-1-azoniabicyclo[2.2.2]octane (hereinafter indicated as compound 1) is a novel compound which has been disclosed in the co-pending patent Application no. PCT/EP2007/057585, incorporated herein by reference. The compound 1 has the following chemical structure:

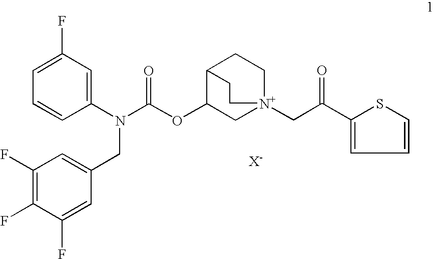

wherein X′ is a pharmaceutically acceptable anion preferably selected from the group consisting of chloride, bromide, iodide, sulfate, phosphate, methanesulfonate, nitrate, maleate, acetate, citrate, fumarate, tartrate, oxalate, succinate, benzoate, and p-toluenesulfonate.

### SUMMARY OF THE INVENTION

Accordingly, it is one object of the present invention to provide novel dry powder formulations suitable to be administered by inhalation by means of a dry powder inhaler which are useful for the prevention and/or treatment of an inflammatory or obstructive airways disease such as asthma and COPD.

It is another object of the present invention to provide novel processes for the preparation of such a formulation.

It is another object of the present invention to provide novel inhalers which contain such a formulation.

It is another object of the present invention to provide novel processes for preparing such an inhaler.

It is another object of the present invention to provide novel methods for the prevention and/or treatment of an inflammatory or obstructive airways disease such as asthma and COPD by administering such a formulation.

These and other objects, which will become apparent during the following detailed description, have been achieved by the inventors' discovery that pharmaceutical formulations in the form of inhalable dry powder comprising micronized particles of a pharmaceutically acceptable salt of 3-[[[(3-fluorophenyl)[(3,4,5-trifluoro phenyl)methyl]amino]carbonyl]oxy]-1-[2-oxo-2-(2-thienyl)ethyl]-1-azoniabicyclo [2.2.2]octane (compound 1) as active ingredient, and particles of a physiologically acceptable pharmacologically-inert solid carrier are effective for the prevention and/or treatment of an inflammatory or obstructive airways disease such as asthma and COPD by administering such a formulation.

Thus, in a first embodiment, the present invention provides a pharmaceutical formulation in the form of inhalable dry powder comprising micronized particles of a pharmaceutically acceptable salt of 3-[[[(3-fluorophenyl)[(3,4,5-trifluoro phenyl)methyl]amino]carbonyl]oxy]-1-[2-oxo-2-(2-thienyl)ethyl]-1-azoniabicyclo [2.2.2]octane (compound 1) as active ingredient, and particles of a physiologically acceptable pharmacologically-inert solid carrier.

In another embodiment, the present invention provides a dry powder inhaler comprising with said inhalable dry powder of the present invention.

In another embodiment, the present invention also relates to the use of the inhalable dry powder formulation described before as a medicament.

In a further embodiment, the present invention provides the use of the inhalable dry powder described before for the prevention and/or treatment of an inflammatory or obstructive airways disease such as asthma or chronic obstructive pulmonary disease (COPD).

In yet a still further embodiment, the present invention provides a method of preventing and/or treating an inflammatory or obstructive airways disease such as asthma or chronic obstructive pulmonary disease (COPD), which comprises administration by inhalation of an effective amount of the inhalable dry powder described before.

In another embodiment, the present invention provides a process for making such a pharmaceutical formulation.

In another embodiment, the present invention provides a process for making a dry powder inhaler which contains such a pharmaceutical formulation.

Finally, the present invention provides packages which comprise an inhalable dry powder formulation described before and a dry powder inhaler.

In particular the chloride salt of compound 1, has been found to be equieffective to tiotropium bromide in terms of receptor potency and duration of action, but significantly short-acting on the M2 receptors.

Therefore compound 1 may provide significant therapeutic benefit in the treatment of respiratory diseases such as asthma and COPD, when administered by inhalation.

Antimuscarinic drugs could be administered to the respiratory tract by inhalation in the form of dry powder by means of suitable inhalers known as dry powder inhalers (DPIs).

Thus, an aim of the present invention is to provide an inhalable dry powder composition that comprise a pharmaceutically acceptable salt of 3-[[[(3-fluorophenyl)[(3,4,5-trifluoro phenyl)methyl]amino]carbonyl]oxy]-1-[2-oxo-2-(2-thienyl)ethyl]-1-azoniabicyclo [2.2.2]octane (compound 1) as active ingredient.

Optimally said formulation shall exhibit good flowability, good uniformity of distribution of the active ingredient and adequate chemical and physical stability in the device before use. It shall also give rise to a good respirable fraction as well as deliver an accurate therapeutically active dose of the active ingredient.

### DETAILED DESCRIPTION OF THE PREFERRED EMBODIMENTS

In the context of the present invention, the terms “active drug”, “active ingredient”, “active” and “active substance”, “active compound”, and “therapeutic agent” are synonymous and used interchangeably.

The terms “muscarinic receptor antagonists”, “antimuscarinic drugs”, and “anticholinergic drugs” are synonymous and are used interchangeably.

As used herein the term “substantially optically pure” means an active ingredient having an optical purity higher than 95% w/w, preferably higher than 98% w/w.

By “daily therapeutically effective dose” it is meant that thee quantity of active ingredient administered at one time by inhalation upon actuation of the inhaler. Said daily dose may be delivered in one or more actuations, preferably one actuation (shot) of the inhaler.

For “actuation” it is meant the release of active ingredient from the device by a single activation (e.g. mechanical or breath).

In general terms, the particle size of particles is quantified by measuring a characteristic equivalent sphere diameter, known as volume diameter, by laser diffraction. The particle size can also be quantified by measuring the mass diameter by means of suitable instrument well known to the skilled person such as, for instance the sieve analyser.

The volume diameter (VD) is related to the mass diameter (MD) by the density of the particles (assuming a size independent density for the particles).

In the present application, the particle size is expressed in terms of mass diameter and the particle size distribution is expressed in terms of: i) the mass median diameter (MMD) which corresponds to the diameter of 50 percent by weight or volume respectively, of the particles, and ii) the MD in micron of 10% and 90% of the particles, respectively.

As used herein the term “good flowability” refers to a formulation that is easy handled during the manufacturing process and is able of ensuring an accurate and reproducible delivering of the therapeutically effective dose. Flow characteristics can be evaluated by measuring the Carr's index; a Carr's index of less than 25 is usually taken to indicate good flow characteristics.

As used herein, the expression “good homogeneity” refers to a formulation wherein, upon mixing, the content uniformity of the active ingredient, expressed as relative standard deviation (RSD), is less than 5%.

As used herein, the expression “chemically stable” refers to a formulation that meets the requirements of the ICH Guideline Q1A referring to “Stability Testing of new Active Substances (and Medicinal Products)”.

As used herein, the expression “physically stable in the device before use” refers to a formulation wherein the active particles do not substantially segregate and/or detach from the surface of the carrier particles during fabrication of the dry powder and in the delivery device before use. The tendency to segregate can be evaluated according to Staniforth et al., J. Pharm. Pharmacol., 34,700-706, 1982 and it is considered acceptable if the distribution of the active ingredient in the powder formulation after the test, expressed as relative standard deviation (RSD), does not change significantly with respect to that of the formulation before the test.

As used herein, the expression “respirable fraction” refers to an index of the percentage of active particles which would reach the deep lungs in a patient. The respirable fraction, also termed fine particle fraction, is evaluated using a suitable in vitro apparatus such as Multistage Cascade Impactor or Mutli Stage Liquid Impinger (MLSI) according to procedures reported in common Pharmacopeias. It is calculated by the ratio between the respirable dose and the delivered dose. The delivered dose is calculated from the cumulative deposition in the apparatus, while the respirable dose (fine particle dose) is calculated from the deposition on Stages 3 (S3) to filter (AF) corresponding to particles ≦4.7 microns. A respirable fraction higher than 30% is an index of good inhalatory performances.

As used herein, the expression “accurate therapeutically active dose of the active ingredient” refers to a formulation wherein the variation between the mean delivered daily dose and the mean emitted dose is equal to or less than 15%, preferably less than 10%.

Thus, in a first embodiment, the present invention provides novel pharmaceutical formulations in the form of inhalable dry powder comprising micronized particles of a pharmaceutically acceptable salt of 3-[[[(3-fluorophenyl)[(3,4,5-trifluoro phenyl)methyl]amino]carbonyl]oxy]-1-[2-oxo-2-(2-thienyl)ethyl]-1-azoniabicyclo [2.2.2]octane (compound 1) as active ingredient, and particles of a physiologically acceptable pharmacologically-inert solid carrier (hereinafter the carrier).

Compound 1 has the following chemical structure:

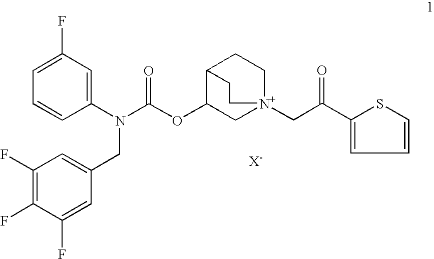

wherein the anion X′ is selected for the group consisting of chloride, bromide, iodide, sulfate, phosphate, methanesulfonate, nitrate, maleate, acetate, citrate, fumarate, tartrate, oxalate, succinate, benzoate, and p-toluenesulfonate. Compound 1 is preferably used in the form of its chloride salt.

It will be apparent to those skilled in the art that compound 1 displays an asymmetric carbon on the quinuclidine ring and hence may be in the form of a mixture of two optical stereoisomers, (3R)- and (3S)-stereoisomers. In the preferred embodiments compound 1 is used in the form of substantially pure (3R)-enantiomer. The (3R)-enantiomer of compound 1 in the form of chloride salt is hereinafter referred to as compound 1′.

The compositions according to the present invention comprise the active ingredient in an amount such that, in case of administration by inhalation from inhalers, the daily therapeutically effective dose (hereinafter the daily dose) of compound 1 is advantageously comprised between about 0.1 μg and about 80 μg, preferably between about 0.5 μg and about 40 μg and even more preferably between about 1 and about 20 μg. Said dose will depend on the kind and the severity of the disease and the conditions (weight, sex, age) of the patient and shall be administered one or more times a day, preferably once a day.

In one embodiment, the daily dose may be reached by a single or double administration.

In another preferred embodiment, the daily dose may be reached by a single administration and delivered in one actuation of the inhaler.

In another preferred embodiment, the daily dose may be reached by a single administration and delivered in more actuations of the inhaler, preferably two.

In another preferred embodiment, the daily dose may be reached by a double administration and delivered in one actuation of the inhaler.

In another preferred embodiment, the daily dose may be reached by a double administration and delivered in more actuations of the inhaler, preferably two.

In one embodiment, the daily dose of a pharmaceutical composition comprising compound 1′ is comprised between 1 μg and 20 μg, preferably between 1 μg and 10 μg and more preferably between 1 μg and 5 μg.

The quantities of active substance in the compositions which are administered per single dose can be calculated analogously if instead of the chloride salt of compound 1, another salt is used.

The particles of the salt of compound 1 in the formulation according to the present invention must be in a finely divided (micronized) form, i.e. their mass median diameter should generally be equal to or less than 10 micron, preferably less than 6 micron, more preferably comprised between 1 and 6 micron. The active ingredient may be produced in the desired particle size using methods known to those skilled in the art, e.g. milling, direct precipitation, spray-drying, freeze-drying or supercritical fluids.

The carrier particles may be made of any physiologically acceptable pharmacologically-inert material or combination of materials suitable for inhalatory use. For example, the carrier particles may be composed of one or more materials selected from sugar alcohols; polyols, for example sorbitol, mannitol and xylitol, and crystalline sugars, including monosaccharides and disaccharides; inorganic salts such as sodium chloride and calcium carbonate; organic salts such as sodium lactate; and other organic compounds such as urea, polysaccharides, for example starch and its derivatives; oligosaccharides, for example cyclodextrins and dextrins.

Advantageously, the carrier particles are made of a crystalline sugar, for example, a monosaccharide such as glucose or arabinose, or a disaccharide such as maltose, saccharose, dextrose or lactose. Preferably, the carrier particles are made of lactose, more preferably of alpha-lactose monohydrate.

In one embodiment, the invention the powder formulation may be in form of agglomerated spheronized particles, also known as soft pellets, wherein the particles of the salt of compound 1 and the particles of the carrier are both in a finely divided form, i.e. their mass median diameter is generally less than 10 micron, preferably from 1 to 6 micron. Said kind of formulations may be prepared according to methods known to the skilled person. Generally the process comprises the steps of:

i) micronising together the active ingredient and the carrier; and

ii) subjecting the resulting co-micronized mixture to agglomeration and spheronisation.

Alternatively, the process comprises the following steps:

i) micronising separately the active ingredient and the carrier;

ii) mixing the micronized components; and

iii) subjecting the resulting mixture to agglomeration and spheronisation.

In another embodiment of the present invention, the formulation comprises coarse particles of a carrier together with the drug in the finely divided form, a type of formulation known in the art as ordered mixture. Advantageously, said coarse carrier particles have a mass diameter (MD) of at least 50 microns, more advantageously greater that 90 microns. Preferably the MD is between 50 micron and 500 micron. In certain embodiments of the present invention, the MD of the coarse carrier particles is between 90 and 150 micron. In other embodiments, the MD of the coarse carrier particle is between 150 and 400 micron, and preferably between 210 and 355 micron. When their MD is comprised between 150 and 400 micron, the coarse carrier particles have preferably a relatively highly fissured surface, that is, on which there are clefts and valleys and other recessed regions, referred to herein collectively as fissures.

The “relatively highly fissured” coarse particles can be defined in terms of fissure index or rugosity coefficient as described in WO 01/78695 and WO 01/78693, incorporated herein by reference in their entireties, and they can be characterized according to the description therein reported.

Said coarse carrier particles may also be characterised in terms of tapped density or total intrusion volume measured as reported in WO 01/78695, incorporated herein by reference in its entirety. The tapped density of the coarse carrier particles is advantageously less than 0.8 g/cm³, preferably between 0.8 and 0.5 g/cm³. The total intrusion volume is of at least 0.8 cm³ preferably at least 0.9 cm³.

When the formulation of the present invention is in the form of the aforementioned ordered mixture, it may advantageously comprise an additive material able to promote the release of the active particles from the carrier particles on actuation of the inhaler device, and hence able of improving the respirable fraction. The additive material, which is preferably bound to the surface of the coarse carrier particles, is of a different material from the carrier particles. Advantageously, the additive material is an amino acid, preferably selected from the group consisting of leucine, isoleucine, lysine, valine, methionine, phenylalanine. The additive may be a salt of a derivative of an amino acid, for example aspartame or acesulfame K. In one embodiment of the present invention the additive particles consist substantially of leucine, advantageously L-leucine.

Alternatively, the additive material may include or consist of one or more water soluble surface active materials, for example lecithin, in particular soya lecithin.

In a particular embodiment of the present invention, the additive material may include or consist of one or more lubricants selected from the group consisting of stearic acid and salts thereof such as magnesium stearate, sodium lauryl sulphate, sodium stearyl fumarate, stearyl alcohol, and sucrose monopalmitate.

Other possible additive materials include talc, titanium dioxide, aluminium dioxide, and silicon dioxide.

Advantageously, at least 90% by weight of the additive particles has a mass diameter (MD) of less than 35 microns. Advantageously, the MMD of the additive particles is not more than 25 micron, preferably not more than 15 micron, and more preferably not more than 10 micron.

The optimum amount of additive material shall depend on the chemical composition and other properties of the additive material. In general, the amount of additive shall be not more than 10% by weight, based on the total weight of the formulation. However, it is thought that for most additives the amount of additive material should be not more than 5%, preferably not more than 2% or even not more than 1% by weight or not more than 0.5% based on the total weight of the formulation. In general, the amount of additive material is of at least 0.01% by weight based on the total weight of the formulation.

In one of the preferred embodiment of the invention, the additive material is magnesium stearate. The amount of magnesium stearate is generally comprised between 0.01 and 2%, preferably between 0.02 and 1%, more preferably between 0.1% and 0.5% by weight based on the total weight of the formulation.

Magnesium stearate may cover the surface of the carrier particles in such a way as that the extent of the molecular surface coating is at least of 5%, preferably more than 10%, more preferably more than 15%, even more preferably equal to or more than and 25%. The extent of molecular surface coating, which indicates the percentage of the total surface of the carrier particles coated by magnesium stearate, may be determined by water contact angle measurement as reported in WO 00/53157, incorporated herein by reference in its entirety, or by electron scanning microscope.

The formulations of the present invention, when in the form of ordered mixture, may also comprise fine particles of a physiologically acceptable pharmacologically-inert material with a mass median diameter (MMD) equal to or less than 10 microns. The percentage of fine particles of physiologically acceptable pharmacologically-inert material is advantageously comprised between 0.1 and 40% of the total amount of the formulation. Preferably, the coarse particles and the fine particles are constituted of the same physiologically acceptable pharmacologically-inert material.

In a particularly preferred embodiment of the invention, a formulation analogous to the teaching of WO 01/78693 is provided, said formulation comprising:

i) particles of a salt of compound 1 in a micronized form;

ii) a fraction of microparticles constituted of a mixture composed of particles of physiologically acceptable pharmacologically-inert material and particles of an additive material, said microparticles having a MMD equal to or less than 10 microns; and

iii) a fraction of particles of a physiologically acceptable pharmacologically-inert material having a mass diameter (MD) comprised between 150 micron and 400 microns, preferably between 212 and 355 microns.

Advantageously, the fraction of microparticles is composed of 90 to 99.5% by weight of the physiologically acceptable pharmacologically-inert material and 0.5 to 10% by weight of the additive material, and the ratio between the fraction of microparticles and the fraction of coarse particles is comprised between 1:99 and 40:60% by weight, preferably between 5:95 and 30:70% by weight, even more preferably between 10:90 and 20:80% by weight. Preferably the physiologically acceptable inert material is α-lactose monohydrate, and the additive material is magnesium stearate.

In a more preferred embodiment, the fraction of microparticles is composed of 98 to 99% by weight of α-lactose monohydrate and 1 to 2% by weight of magnesium stearate and the ratio between the fraction of microparticles and the fraction of coarse particles made of α-lactose monohydrate is 10:90% by weight, respectively.

The amount of magnesium stearate in the final formulation is advantageously comprised between 0.01 and 1.0% by weight, preferably between 0.05 and 0.5% by weight, more preferably between 0.1 and 0.4% by weight on the total weight of the formulation.

Magnesium stearate is added to said formulation with the aim of improving the respirable fraction of the active ingredient.

The formulation in form of ordered mixture according to the invention may be prepared according to methods well known to the skilled person. Said methods comprise mixing together the coarse carrier particles, the optional fine carrier particles, and the additive particles, and finally adding the finely divided pharmaceutically active compound to the resulting mixture.

The particularly preferred formulation according to the invention may be prepared according to the methods reported in WO 01/78693, which is incorporated herein by reference in its entirety. Among the methods therein described, the formulation is preferably prepared according to a process which comprises the following steps:

a) preparing microparticles constituted of a mixture composed of particles made of a physiologically acceptable pharmacologically-inert material and particles of the additive, the inert material and the additive being first-mixed together and then co-micronised;

b) mixing the microparticles of step a) with coarse particles of a physiologically acceptable pharmacologically-inert material such that microparticles adhere to the surface of the coarse particles; and

c) adding by mixing the active particles in the micronized form to the particles of step b).

The co-micronization step may be carried out by methods known to the skilled person such as those reported in WO 02/00197, incorporated herein by reference in its entirety. Preferably said step is carrier out by milling, more preferably by using a jet mill according to the conditions reported in WO 01/78693, which is incorporated herein by reference in its entirety.

Advantageously during the step a) the additive may be embedded in the formed microparticles, or alternatively, in the case of a lubricant such as magnesium stearate, the additive may coat the surface of the carrier particles in such a way as that the extent of molecular surface coating is at least of 5%, preferably more than 10%, more preferably more than 15%, even more preferably more than and 35%. The extent of molecular surface coating indicates the percentage of the total surface of the carrier particles coated by magnesium stearate.

The presence of the additive material embedded in the microparticles may be detected according to methods known to the person skilled in the art, for instance, by electron scanning microscope coupled to microcalorimetry. Alternatively, as reported above, the extent of molecular surface coating may be determined by water contact angle measurement as reported in WO 00/53157 or by electron scanning microscope.

The formulations of the present invention may further comprise other therapeutic agents useful for the prevention and/or treatment of a respiratory disease, e.g. corticosteroids such as budesonide and its epimers, beclometasone dipropionate, triamcinolone acetonide, fluticasone propionate, flunisolide, mometasone furoate, rofleponide and ciclesonide, anticholinergic or antimuscarinic agents such as ipratropium bromide, oxytropium bromide, tiotropium bromide, glycopyrrolate bromide, and the group of phosphodiesterase-4 (PDE-4) inhibitors such as roflumilast, and their combinations.

The dry powder formulation herein described may be used in any customary dry powder inhalers. Advantageously said formulation is filled in a multidose dry powder inhaler comprising a powder reservoir such as that described in WO 2004/012801, incorporated herein by reference in its entirety.

Administration of the formulations of the present invention may be indicated for prevention and/or the treatment of mild, moderate or severe acute or chronic symptoms or for prophylactic treatment of an inflammatory or obstructive airways disease such as asthma and chronic obstructive pulmonary disease (COPD). Other respiratory disorders characterized by obstruction of the peripheral airways as a result of inflammation and presence of mucus such as chronic obstructive bronchiolitis and chronic bronchitis may also benefit from the formulation of the invention.

Other features of the invention will become apparent in the course of the following descriptions of exemplary embodiments which are given for illustration of the invention and are not intended to be limiting thereof.

### EXAMPLES

### Example 1

### Inhalable Dry Powder Formulations Comprising Compound 1′

A powder formulation according to the invention is prepared with the composition reported in Table 1:

TABLE 1

| | Amounts | | |
|---|---|---|---|
| | Per shot of | | |
| | the inhaler | | Daily dose |
| Components | mg | % | μg |
| Compound 1′ | 0.01 | 0.1 | 10 |
| alpha-lactose monohydrate 212-355 μm | 8.99 | 89.91 | |
| Pre-blend | 0.99 | 9.99 | |
| Total weight | 10 | | |

The final formulation is filled in the multidose dry powder inhaler described in WO 2004/012801.

The aerosol performances of said formulation are evaluated using a Multi Stage Liquid Impinger (MSLI) according to the procedure described in European Pharmacopoeia 2^{nd} edition, 1995, part V. 5.9.1, pages 15-17.

Further powder formulations according to the invention are prepared with the compositions reported in Tables 2 and 3.

TABLE 2

| | Amounts | | |
|---|---|---|---|
| | Per shot of | | |
| | the inhaler | | Daily dose |
| Components | mg | % | μg |
| Compound 1′ | 0.02 | 0.2 | 20 |
| alpha-lactose monohydrate 90-150 μm | 9.955 | 99.55 | |
| magnesium stearate | 0.025 | 0.25 | |
| Total weight | 10 | | |

TABLE 3

| | Amounts | | |
|---|---|---|---|
| | Per shot of | | |
| | the inhaler | | Daily dose |
| Components | mg | % | μg |
| Compound 1′ | 0.005 | 0.05 | 5 |
| alpha-lactose monohydrate 90-150 μm | 9.97 | 99.7 | |
| magnesium stearate | 0.025 | 0.25 | |
| Total weight | 10 | | |

### Example 2

### Assessment of the Bronchodilation Activity of Compound 1′

Airway reactivity is measured using barometric plethysmography (Buxco, USA). Male guinea pigs (500-600 g) are individually placed in plexiglass chambers. After an acclimatisation period, animals are exposed to nebulised saline for 1 minute to obtain airway baseline reading. This is followed by a 1 minute challenge with nebulised acetylcholine (Ach)-2.5 mg/mL. After 60 minutes, 5 minute nebulisation of vehicle or the compound I′ in the range 2.5-250 μM are applied, and Ach challenge is then repeated after 2, 5, 24, 48 and 72 hours (h). Recording of pressure fluctuations in the chambers are taken for 5 minutes after each nebulisation and analysed to calculate Enhanced Pause (Penh). Airway reactivity is expressed as percentage increase in Penh compared with Penh values from the nebulisation of vehicle.

Two hours after the end of nebulisation with compound 1′, the Ach-induced increase in Penh is dose-dependently inhibited by the compound, with a maximal effect of 99.6±0.4 at 50 μM.

As for the time-course of the effect, compound 1′ shows increasing duration of action with increasing dose.

After inhalation of 250 μM of compound I′, effect persists unchanged up to 48 hours (83.0±16.1%), while at 72 hours a residual activity of 34.8±20.9% is present. Twenty-four hours after 25 and 50 μM compound 1′ inhalation, a significant bronchoprotective effect was observed (63.7±15.1% and 87.1±8.7%, respectively). At 50 μM, a significant inhibition persists up to 48 hours (49.2±23.2%). Inhalation of lower concentrations results in an effect that did not exceed the 5 hour observation point.

The estimation of lung levels of compound 1′ achieved after nebulisation endowed with a submaximal bronchodilator activity at 2 hours after treatment reveals that the its retained dose in the target organ is about 50 μg/kg. If an extrapolation of these results from guinea pig to human is made, it can be predicted that in patients the daily dose might be comprised between 1 and 20 μg, preferably between 1 and 10 μg and more preferably between 1 and 5 μg.

Where a numerical limit or range is stated herein, the endpoints are included. Also, all values and subranges within a numerical limit or range are specifically included as if explicitly written out.

Obviously, numerous modifications and variations of the present invention are possible in light of the above teachings. It is therefore to be understood that, within the scope of the appended claims, the invention may be practiced otherwise than as specifically described herein.

All patents and other references mentioned above are incorporated in full herein by this reference, the same as if set forth at length.

1. An inhalable dry powder formulation, comprising particles of a pharmaceutically acceptable salt of 3-[[[(3-fluorophenyl)[(3,4,5-trifluoro phenyl)methyl]amino]carbonyl]oxy]-1-[2-oxo-2-(2-thienyl)ethyl]-1-azoniabicyclo [2.2.2]octane, and particles of a carrier made of a physiologically acceptable pharmacologically-inert material.

2. The inhalable powder according to claim 1, wherein said salt is selected from the group consisting of chloride, bromide, iodide, sulfate, phosphate, methanesulfonate, nitrate, maleate, acetate, citrate, fumarate, tartrate, oxalate, succinate, benzoate, and p-toluenesulfonate.

3. The inhlable powder according to claim 1, wherein said salt is the chloride salt of the (3R)-enantiomer.

4. The inhalable powder according to claim 3, wherein said chloride salt of the (3R)-enantiomer of 3-[[[(3-fluorophenyl)[(3,4,5-trifluoro phenyl)methyl]amino]carbonyl]oxy]-1-[2-oxo-2-(2-thienyl)ethyl]-1-azoniabicyclo [2.2.2]octane is present in an amounts suitable for administration of a daily dose of about 1 μg to about 20 μg.

5. The inhalable powder according to claim 3, wherein said chloride salt of the (3R)-enantiomer of 3-[[[(3-fluorophenyl)[(3,4,5-trifluoro phenyl)methyl]amino]carbonyl]oxy]-1-[2-oxo-2-(2-thienyl)ethyl]-1-azoniabicyclo [2.2.2]octane is present in an amounts suitable for administration of a daily dose of about 1 μg to about 10 μg.

6. The inhalable powder according to claim 3, wherein said chloride salt of the (3R)-enantiomer of 3-[[[(3-fluorophenyl)[(3,4,5-trifluoro phenyl)methyl]amino]carbonyl]oxy]-1-[2-oxo-2-(2-thienyl)ethyl]-1-azoniabicyclo [2.2.2]octane is present in an amounts suitable for administration of a daily dose of about 1 μg to about 5 μg.

7. The inhalable powder according to claim 1, wherein said carrier comprises a crystalline sugar selected from the group consisting of glucose, arabinose, maltose, saccharose, dextrose, and lactose or a polyalcohol selected from the group consisting of mannitol, maltitol, lactitol, and sorbitol.

8. The inhalable powder according to claim 7, wherein said carrier comprises lactose.

9. The inhalable powder according to claim 8, wherein said carrier comprises α-lactose monohydrate.

10. The inhalable powder according to claim 1, wherein said carrier is in the form of finely divided particles having a mass median diameter (MMD) equal to or of less than 10 microns.

11. The inhalable powder according to claim 1, wherein said carrier is in the form of coarse particles having a mass diameter of at least 50 microns.

12. The inhalable powder according to claim 11, wherein the mass diameter is greater than 90 microns.

13. The inhalable powder according to claim 12, wherein the mass diameter is 150 to 400 micron.

14. The inhalable powder according to claim 1, wherein said carrier comprises a mixture of coarse particles having a mass diameter greater than 90 microns and finely divided particles with a MMD equal to or less than 10 microns.

15. The inhalable powder according to claim 10, further comprising one or more additive materials selected form the group consisting of an amino acid, a water-soluble surface active agents, a lubricants, a glidant, and mixtures thereof.

16. The inhalable powder according to claim 15, which comprises leucine.

17. The inhalable powder according to claim 15, which comprises magnesium stearate.

18. The inhalable powder according to claim 17, which comprises magnesium stearate in an amount of 0.01 to 2% by weight, based on the total weight of the formulation.

19. The inhalable powder according to claim 18, which comprises magnesium stearate in an amount of 0.02 and 1% w/w, based on the total weight of the formulation.

20. A dry powder inhaler, which contains an inhalable dry powder formulation according to claim 1.

21. A method for the prevention and/or treatment of a respiratory disease, comprising administering an effective amount of an inhalable dry powder formulation according to claim 1 to a subject in need thereof.

22. The method of claim 21, wherein said respiratory disease is asthma or chronic obstructive pulmonary disease.

23. A package, comprising an inhalable dry powder formulation according to claim 1 and a dry powder inhaler.
